(19) United States
(12) Patent Application Publication
(10) Pub. No.: US 2024/0180557 A1
(43) Pub. Date: Jun. 6, 2024

(54) MINIMALLY INVASIVE APPENDECTOMY DEVICE AND METHOD

(71) Applicant: Wake Forest University Health Sciences, Winston-Salem, NC (US)

(72) Inventors: James A. Smith, Winston-Salem, NC (US); James E. Johnson, Winston-Salem, NC (US)

(21) Appl. No.: 18/287,491

(22) PCT Filed: Apr. 23, 2021
(86) PCT No.: PCT/US21/28786

§ 371 Date: Oct. 19, 2023

(51) Int. Cl. A61B 17/12; A61B 17/00

(52) U.S. Cl. CPC A61B 17/12013; A61B 2017/00823; A61B 2017/12018

(57) ABSTRACT

Disclosed are methods and devices for minimally-invasive appendectomies. The methods may comprise inserting an inverter apparatus into an interior cavity of the appendix of a subject and inverting the appendix, closing the base portion of the inverted appendix with the one or more closure devices, separating the inverted appendix from the cecum with a separation apparatus positioned at the base portion of the inverted appendix adjacent to the one or more closure devices, and removing the separated appendix from the subject through the colon.

### FIELD

The presently disclosed subject matter relates to methods and devices for managing appendages in a patient subject. In certain embodiments, the methods and devices may be employed for removal of the appendix of a patient subject.

### BACKGROUND

The appendix is a finger-shaped, blind-ended tube that is part of the gastrointestinal (GI) tract. The GI tract includes an upper portion and a lower portion. The lower GI tract includes most of the small intestine and the large intestine, which includes the colon, rectum, and anal canal. The appendix is attached to the cecum, a small pouch considered to be the beginning of the large intestine. The appendix is open to the GI tract and may be susceptible to blockage and infection. The infection or obstruction can cause bacteria in the appendix to grow out of control, leading to appendicitis. Depending on the severity of the appendicitis, surgical removal of the appendix or appendectomy, may be necessary to treat the infection. Appendicitis is a common condition that affects approximately 7% of the US population.

Appendectomies are one of the more common emergency surgical procedures. During an open appendectomy, an incision about 2 to 4 inches in length may be made in the lower right side of the abdomen, and the appendix removed through the incision. To access the appendix, the abdominal muscles need to be separated. During a less invasive laparoscopic appendectomy, multiple small incisions may be made and a laparoscope used to remove the appendix through one of the incisions. Appendectomies can be expensive and may present additional risk to the patient. For acute or severe appendicitis, an appendectomy is the standard of care.

### SUMMARY

Described herein are methods and devices for management of the appendix that can engage and invert the appendix for excision and removal as part of a minimally invasive procedure.

In some embodiments, a method for management of the appendix may comprise inserting an inverter apparatus into the colon through cecum of a subject to reach the appendix, inverting the appendix, positioning one or more closure devices around a base portion of the inverted appendix, closing the base portion of the inverted appendix with the one or more closure devices, positioning a separation apparatus at the base portion of the inverted appendix adjacent to the one or more closure devices, separating the inverted appendix from the cecum with a separation apparatus, and removing the separated appendix from the subject. The method may include engaging the inverter apparatus with an interior wall of the appendix and pulling the inverter apparatus into the cecum to invert the appendix. In certain embodiments, the base of the inverted appendix may be substantially aligned with an outer wall of the cecum. Optionally, the method can further comprise reducing the size of the inverted appendix to facilitate removal of the inverted appendix from the subject. In some embodiments, a size reduction apparatus may be positioned around the inverted appendix in the cecum and engaged to reduce the size of the inverted appendix.

Also disclosed is a medical device for minimally invasive appendectomies. In some embodiments, a medical device may comprise a head that comprises at least one arm that extends outwardly from a first position of the head, and a handle connected to a second position of the head, where the first position and the second position are opposite one another. In some embodiments, the at least one arm may comprise a hooked, pointed, jagged, or rounded tip. In certain embodiments, the inverter device may include a plurality of arms. In other embodiments, the medical device may comprise a port with negative pressure.

Also disclosed is a kit for minimally invasive appendectomies. In some embodiments, the kit may comprise an inverter apparatus, one or more closure devices, and a separation apparatus. In certain embodiments, the kit may further comprise a size reduction apparatus. Optionally, the kit may include a catheter.

### BRIEF DESCRIPTION OF THE DRAWINGS

FIG. 1 is a schematic showing a front view of the exterior of the intestinal system.

FIG. 2 is a schematic showing a perspective view of the interior of the cecum and appendix.

FIG. 3 is a schematic showing a perspective view of the intestinal system with a catheter scope within the colon.

FIGS. 4A to 4E are schematics showing a side view of the appendix before, during, and after inversion, according to one embodiment described herein.

FIG. 5 is a schematic showing a perspective view of an inverter apparatus in the appendix according to one embodiment described herein.

FIG. 6 is a schematic showing a perspective view of an inverter apparatus in the appendix according to one embodiment described herein.

FIG. 7 is a schematic showing a perspective view of an inverter apparatus with size reduction apparatus in the appendix according to one embodiment described herein.

FIG. 8 is a schematic showing a perspective view of a size reduction apparatus according to one embodiment described herein.

FIGS. 9A to 9C are schematics showing a side view of an inverted appendix during and after closure according to one embodiment described herein.

FIG. 10 is a schematic showing a perspective view of a closure device according to one embodiment described herein.

FIGS. 11A to 11C are schematics showing a side view of an inverted appendix during and after separation according to one embodiment described herein.

FIG. 12 is a schematic showing a perspective view of a separation apparatus according to one embodiment described herein.

FIG. 13 is a schematic showing a perspective view of a separation apparatus according to one embodiment described herein.

FIG. 14 is a schematic showing a perspective view of a separation apparatus according to one embodiment described herein.

### DETAILED DESCRIPTION

The presently disclosed subject matter now will be described more fully hereinafter with reference to the accompanying description and drawings, in which some, but not all embodiments of the presently disclosed subject matter are shown. The presently disclosed subject matter can be embodied in many different forms and should not be construed as limited to the embodiments set forth herein.

Described herein are methods for management of the appendix that can engage and invert the appendix for excision and removal as part of a minimally invasive appendectomy procedure. The methods may use a colonic catheter to engage and invert the appendix, and secure the appendix for excision. The methods for the minimally invasive appendectomy do not require open surgery or laparoscopic surgical procedures to remove the appendix and suture the subject closed. Rather, the methods may use minimally invasive colonic access and procedures used by gastroenterologists in colonoscopies and polypectomies. Colonic access may be gained via the anal canal and a colonic catheter passing into the appendix via the colon and cecum. This approach can allow for lower costs than surgical procedures and the less invasive methods may bring fewer adverse risks than open surgery or laparoscopic surgical procedures.

The present disclosure may be embodied in a variety of ways. In one embodiment, methods of the present disclosure may comprise inserting an inverter apparatus into an interior cavity of the appendix of a subject, engaging the inverter apparatus with an interior wall of the appendix, inverting the appendix by pulling the inverter apparatus into the cecum until the appendix is substantially inverted. In some cases, the base of the inverted appendix may be substantially aligned with an outer wall of the cecum. Once the appendix is substantially inverted, the methods may further comprise positioning one or more closure devices around a base portion of the inverted appendix and closing the base portion of the inverted appendix with the one or more closure devices to prepare the appendix for excision. To excise the appendix, the methods further comprise positioning a separation apparatus at the base portion of the inverted appendix adjacent to the one or more closure devices, separating the inverted appendix from the cecum with the separation apparatus, and removing the separated appendix from the subject. The catheter may include a multi-port head that can accommodate tools such as a camera, irrigation spray, light. The catheter may include an instrument channel that can allow for various tool to be placed through the catheter to reach a target area of the colon or appendix.

Unlike the other appendectomy methods, the methods herein may grasp and invert the appendix inside the cecum, remove the appendix through the colon, with no incision. The appendectomy methods can remove the majority of the appendix from the cecum and substantially prevent infection and appendicitis in high risk patients. The amputated closed stump of the appendix may be rapidly repaved by the host mucosa as a natural surface. The colonic removal of the appendix can provide a long-term solution for managing risk of appendicitis in some patients.

In some embodiments, the inverter apparatus may be a grasping tool that comprises one or more arms. In some cases, the one or more arms may be articulating. Optionally, the arms of the inverter apparatus may comprise a curved tip, a round or jagged tip, hooks, and/or teeth. The tip of the inverter apparatus may engage and hold the interior wall of the appendix for inversion while avoiding penetrating, piercing, or tearing the wall of the appendix. The inverter may include a plurality of arms in certain examples. In other examples, the inverter apparatus may engage with the interior wall of the appendix using negative pressure.

Once the appendix is inverted, the base of the appendage may be sealed or closed. In some embodiments, one or more closure devices may be used to prepare the inverted appendix for excision and to ensure the stump of the excised appendix remains closed. For example, one device may be used, two devices may be used, or three or more devices may be used to close the base of the inverted appendix. In some embodiments, the closure device may comprise at least one of a slip knot, a polymeric zipper tie, a clip, an absorbable elastic band, a suture purse string, a barbed suture, a staple, a strip, or combination thereof. In some examples, the multiple closure devices used may differ or may be identical in kind. In some embodiments, a surgical stapler may be used to place the closure device. In certain embodiments, the surgical stapler may utilize bovine or porcine tissue as a closure device. The closure devices can permit the cecum and colon to function normally, and move food material within the colon once the inverted appendix is excised. In some examples, the closure devices may breakdown and be absorbed by the body. In other examples, the one or more closure devices may remain at the base of the amputated appendix and be quickly covered by proliferation of the host mucosa.

Once the base of the appendix is closed, the inverted appendix may be separated (e.g., cut off). In some cases, the inverter apparatus may remain engaged with the inverted appendix during the separation step. A variety of methods may be used to separate the inverted appendix from the cecum. In some embodiments, the separation apparatus may comprise a metal wire. For example, the separation apparatus may be a metal noose that may lassoed around the base of the inverted appendix and tightened to separate the appendix. In some embodiments, the separation apparatus may be a guillotine-style cutting apparatus. In other embodiments, the separation apparatus may use electromagnetic radiation to separate the inverted appendix. For example, the electromagnetic radiation may be at frequencies between 350-500 kHz. In other examples, the separation apparatus may use electromagnetic radiation at wavelengths between 800-1500 nm. In some embodiments, the separation apparatus may cauterize the inverted appendix. In certain embodiments, ultrasonic vibration or electric current may be used to cut and cauterize the inverted appendix. The separated appendix may remain engaged with the inverter apparatus. In some cases, the separated appendix may be held or contained by the inverter apparatus.

Once separated, the appendix may be removed from subject. In some examples, the appendix may be removed using the inverter apparatus along the pathway of the catheter. By excising the appendix through the colon, the method may eliminate a potential source of infection that can arise from open surgery and laparoscopic incisions of conventional appendectomies.

Optionally, the method may further comprise positioning a size reduction apparatus around the inverted appendix in the cecum prior to removing the inverted appendix from the subject. The size reduction apparatus may be engaged to reduce the size of the appendix to facilitate removal of the inverted appendix from the subject. In some cases, a cross-section of the inverted appendix may be reduced to a width less than a width of catheter. In certain embodiments, a cross-section of the inverted appendix may be reduced to a width of 24 French or less. The size of the separated appendix may be reduced by cutting, crushing, chemical degradation, or other means known by those skilled in the art. In some examples, the size reduction apparatus may comprise articulating arms. Optionally, the size reduction apparatus may further comprise a metallic or polymeric mesh. For example, the arms and/or mesh of the size reduction apparatus may comprise stainless steel, a cobalt chrome alloy, a nickel aluminum alloy, or a nickel titanium alloy, such as Nitinol (available from Memory Corporation, Bethel, CT), or other metal alloy known to those skilled in the art. Optionally, the apparatus may be comprised of a metal alloy coated with a polymer. In certain embodiments, the polymer may be a Polytetrafluoroethylene (PTFE) or GORE-TEX (available from W.L. Gore, Flagstaff, AZ). In some embodiments, the size reduction apparatus may comprise a cutting device. In other embodiments, the size reduction apparatus may comprise degradation chemicals, such as an acid or a base.

In some embodiments, the appendix may be incrementally reduced in size for removal. Portions of the appendix may be separated and removed from the colon. In some cases, portions of the appendix may be separated, reduced in size using a size reduction apparatus, and then removed from the colon.

Illustrative examples are given to introduce the reader to the general subject matter discussed herein and are not intended to limit the scope of the disclosed concepts.

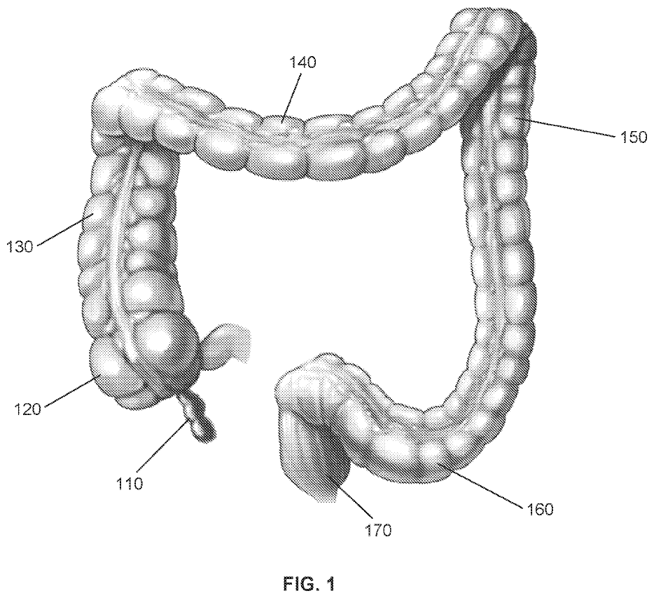

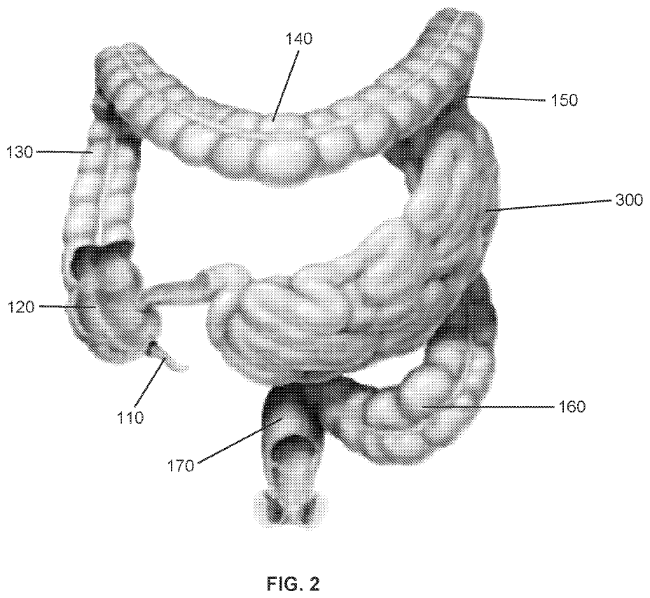

FIG. 1 depicts the exterior of the intestinal system, with the appendix 110 connected to the cecum 120. The cecum 120 is part of the colon 100, which is comprised of the ascending colon 130, transverse colon 140, descending colon 150, sigmoid colon 160, and rectum 170. FIG. 2 shows an perspective view of the colon 100 with cecum 120 connected to the small intestine 300 and appendix 110.

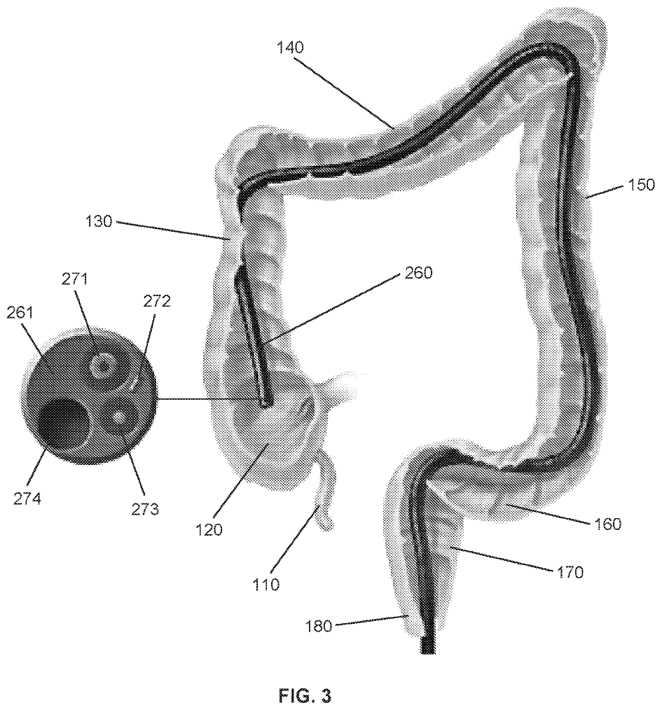

FIG. 3 depicts the positioning of the catheter 260 within the intestinal system. The catheter 260 can be placed in the rectum 170 via the anal canal 180 to access the interior of the colon 100 to reach the cecum 120 and appendix 110. In some examples, the catheter 260 may have a multi-port head 261 and may include a video camera 271, irrigation 272, light 273, and/or an instrument channel 274.

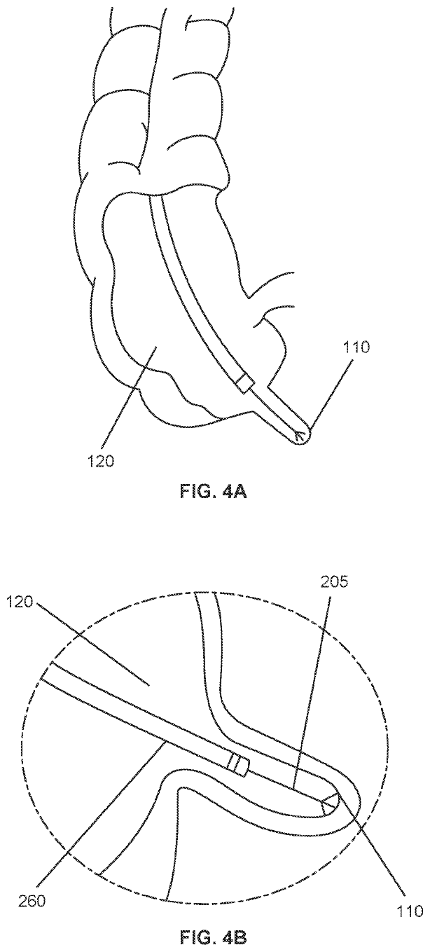

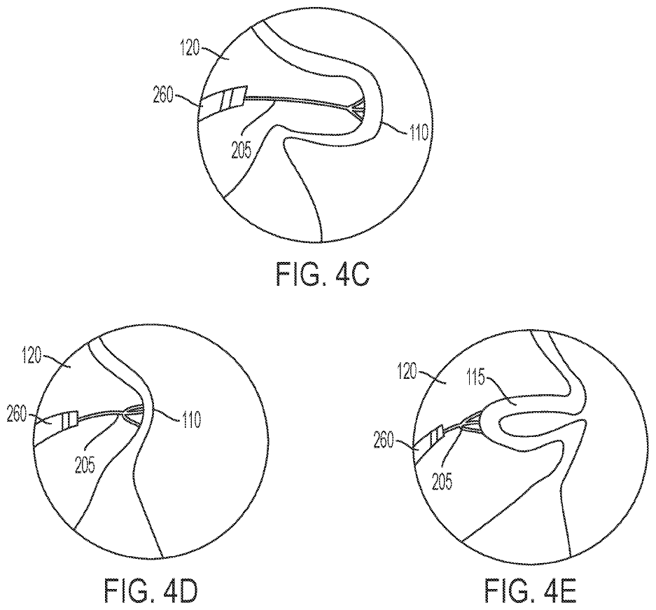

Medical devices, such as an inverter apparatus, can be placed using the catheter 260 to access the cecum 120 and appendix 110. FIG. 4A show the positioning of the catheter 260 within the cecum 120 and appendix 110. FIG. 4B shows the inverter apparatus 205 extending from the catheter 260 into the appendix 110, where the inverter apparatus 205 grasps the wall of the appendix 110.

Once the inverter apparatus grasps the wall of the appendix, the inverter apparatus can be partially retracted from the appendix while remaining engaged with the wall of the appendix, to invert the appendix within the cecum. FIGS. 4B to 4E show the stages of appendix inversion. In FIG. 4B, the inverter apparatus 205 is engaged with the wall of the appendix 110. As shown in FIGS. 4C and 4D, as the inverter apparatus 205 is pulled toward the catheter 260, and the appendix 110 can deform to invert (i.e., turn inside-out). The appendix 110 can continue to deform and invert as the inverter apparatus 205 is pulled in a direction away from the appendix 110. As shown in FIG. 4E, the appendix can be fully inverted 115 inside the cecum 120. The inverter apparatus 205 can remain attached to the portion of the appendix 110 that was the interior wall and becomes the tip of the inverted appendix 115.

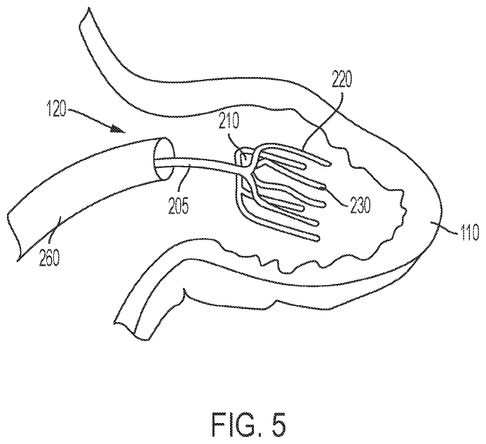

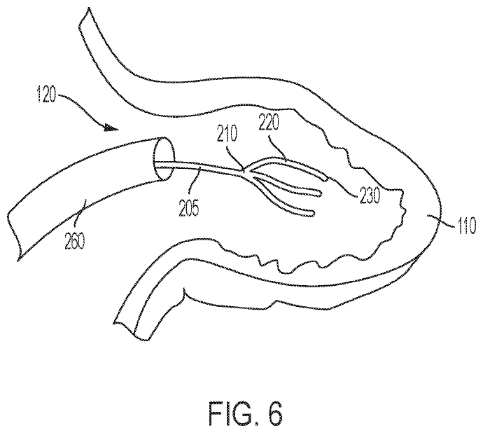

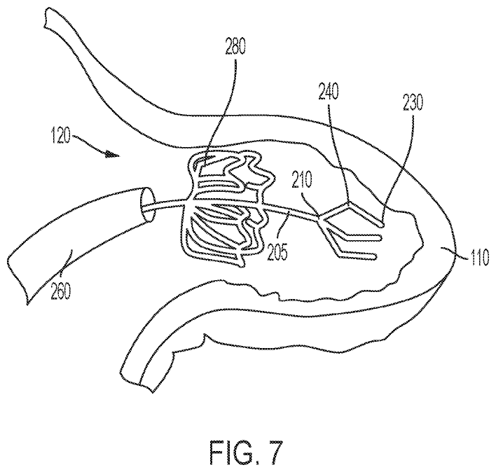

FIGS. 5 to 7 depict various embodiments of the inverter apparatus 205 extending from the catheter 260 through the cecum 120 into the appendix 110. As shown in FIG. 5, the inverter apparatus 205 may comprise a collapsible head 210 the includes multiple arms 220. The arms 220 can include a blunted tip or rounded end feature 230. The head 210 can be in a collapsed state during transport through the catheter 260. Upon exiting the catheter 260, the head 210 may be transitioned from a collapsed state to an open state. In the open state, the head 210 can include a plurality of arms 220 that reach the end of the appendix 110, to the wall of the appendix 110 with the tips 230, and grasp the surface of the appendix 110 with sufficient strength to retain a grasp on the appendix 110 as the inverter apparatus 205 is pulled into the internal guidewire 260 to invert the appendix into the cecum 120. In some cases, the head 210 may be simplified to include only two or three arms 220, as depicted in FIG. 6. In some embodiments, the arms of the inverter apparatus 205 may be articulated. As shown in FIG. 7, the inverter apparatus 205 can include articulated arms 240 that can extend from and contract toward the center of the inverter apparatus 205. The articulation of the arms may aid in obtaining a strong grasp on the appendix 110 for inversion. A size reduction apparatus 280 can be used to reduce the physical size of the appendix for extraction. As shown in the example illustrated in FIG. 7, the size reduction apparatus 280 can be integrated with the inverter apparatus 205. The size reduction apparatus 280 may include a mesh material that can receive the appendix 110 or a portion of the appendix 110 upon inversion and excision. The size reduction apparatus 280 may be collapsible for transport through the catheter 260. Upon exiting the catheter 260, the size reduction apparatus 280 may transition from a collapsed state to an open state ready to receive at least a portion of the inverted appendix.

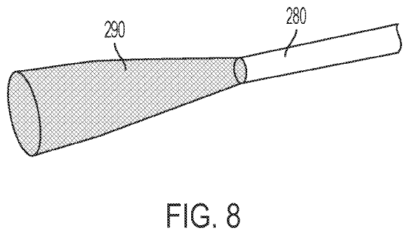

As shown in FIG. 8, one embodiment of the size reduction apparatus 280 may have an elongated or conical opening 290 that can receive at least a portion of the inverted appendix and compress the portion of the appendix to aid in removal of the portion of appendix from the cecum via the internal guide wire through the catheter.

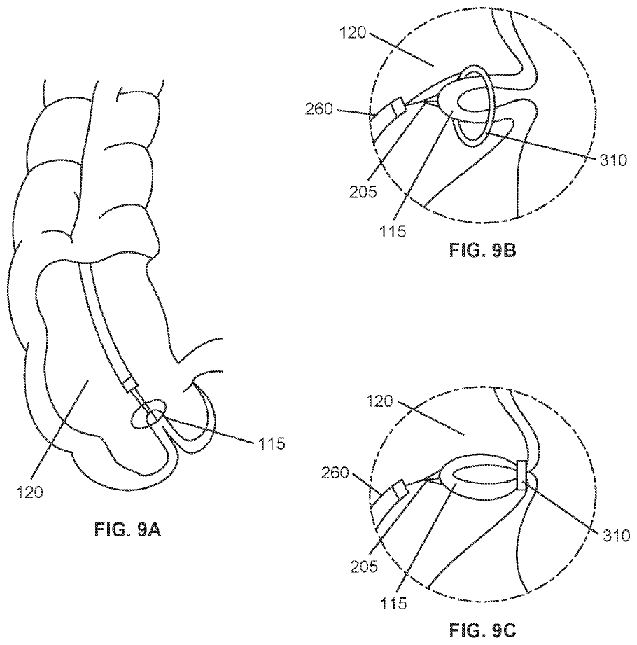

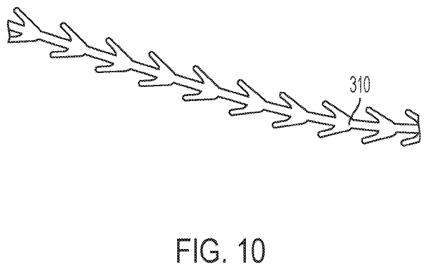

As shown in FIGS. 9A to 9C, once pulled into the cecum 120, the inverted appendix 115 can be closed using one or more closure devices 310. The closure device 310 can be a self-closing tie, a suture, a band, or other closure device. The closure device 310 can slip over the inverted appendix 115, with the inverter apparatus 205 remaining engaged. A barbed suture, shown in FIG. 10, is one example of a closure device 310 that can be used.

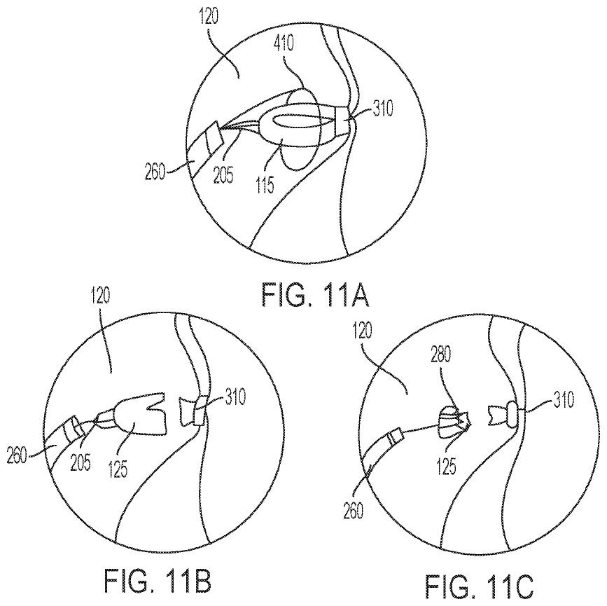

Once the inverted appendix 115 is closed using closure device 310, the tissue of the inverted appendix 115 can be separated, as shown in FIGS. 11A to 11C. The closure device 310 can remain in place during and after the separation of at least a portion of the inverted appendix 115. As shown in FIG. 11A, the separation apparatus 410 can slip over or around the inverted appendix 115, with the inverter apparatus 205 remaining engaged. In some embodiments, once the inverted appendix is separated, the separated appendix 125 may be removed from the colon using the inverter apparatus 205, as shown in FIG. 11B. In some embodiments, the separated appendix 125 may be too large to remove through the catheter. A size reduction apparatus 280 can be used to reduce the size of the excised appendix for removal, as shown in FIG. 11C.

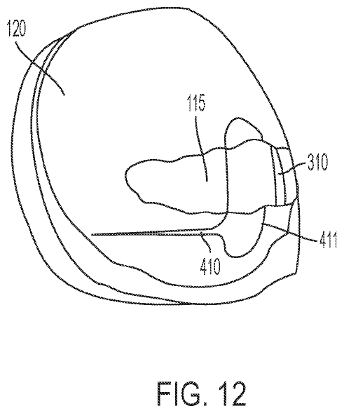

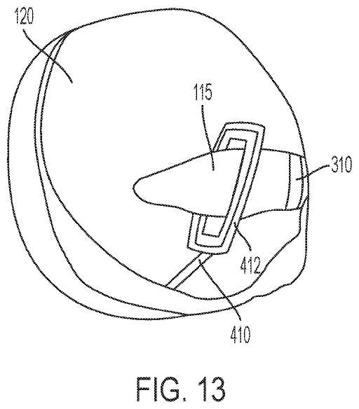

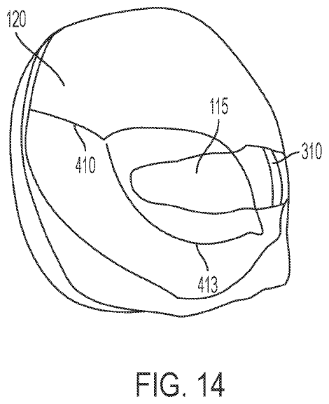

FIGS. 12 to 14 depict various embodiments of the separation apparatus 410 extending into the cecum 120 to separate the inverted appendix 115. As shown in FIG. 12, the separation apparatus 410 may comprise a cutting wire 411. The cutting wire 411 may be placed around the inverted appendix 115 and moved substantially perpendicular to the inverted appendix 115 to cut the inverted appendix 115 and cleave it from the cecum 120. In some cases, the separation apparatus 410 may comprise a guillotine-style cutter 412, as shown in FIG. 13. The cutter 412 may be placed around the inverted appendix 115 and engaged to slice the inverted appendix 115 from the cecum 120. As shown in FIG. 14, the separation apparatus 410 may comprise a cutting device 413. The cutting device 413 may be placed around the inverted appendix 115 and moved substantially perpendicular to the inverted appendix 115 to cut the inverted appendix 115 and cleave it from the cecum 120. In other embodiments, a cutting device may be used to slice the inverted appendix from the side and may not surround the inverted appendix.

In certain embodiments, the inverter apparatus and size reduction apparatus may be integrated in a single apparatus. In other embodiments, the inverter apparatus, size reduction apparatus, and separation apparatus may be integrated into a single apparatus.

In other embodiments, the present disclosure comprises a medical device. The medical device may comprise a head comprising at least one arm extending outwardly from a first position of the head and a handle connected to a second position of the head. In some embodiments, the first position is approximately 180 degrees from the second position. In some embodiments, the at least one arm may comprise a hooked, pointed, jagged, or rounded tip. The head may comprise a plurality of arms in some examples. In some examples, the medical device may further comprise a mesh.

The at least one arm of the medical device may articulate and/or rotate about the head of the device. In certain examples, the device may comprise a plurality of arms that move in a lateral direction at the head. For example, the device may comprise two arms that rotate towards one another and act as a set of pinchers.

The medical device may comprise a metal alloy or polymeric coating. For example, the arms and/or mesh of the size reduction apparatus may stainless steel, a cobalt chrome alloy, a nickel aluminum alloy, or a nickel titanium alloy, such as Nitinol (available from Memory Corporation, Bethel, CT), or other metal alloy known to those skilled in the art. Optionally, the apparatus may be comprised of a metal alloy coated with a polymer. In certain embodiments, the polymer may be a Polytetrafluoroethylene (PTFE) or GORE-TEX (available from W.L. Gore, Flagstaff, AZ).

In some embodiments, the handle may further comprise a lever to articulate the at least one arm. In some cases, the medical device may be an inverter device.

In other embodiments, the present disclosure comprises a kit for minimally-invasive or non-invasive appendectomies, the kit comprising an inverter apparatus, one or more closure devices, and a separation apparatus, as described herein. Optionally, the kit may further comprise a size reduction apparatus. In certain embodiments, the kit may include a colonic catheter.

The terms “a”, “an”, and “the” refer to “one or more” when used in this application, including the claims. Thus, for example, reference to “an arm” includes a plurality of such arms, unless the context clearly is to the contrary (e.g., a plurality of arms), and so forth.

Various embodiments of the disclosure have been described herein. It should be recognized that these embodiments are merely illustrative of the present disclosure. Variations of those preferred embodiments may become apparent to those of ordinary skill in the art upon reading the foregoing description. It is expected that skilled artisans can employ such variations as appropriate, and the disclosure is intended to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated or otherwise clearly contradicted by context.

### Illustrative Embodiments of Suitable Methods and Devices

As used below, any reference to methods, devices, or kits is understood as a reference to each of those methods, devices, or kits disjunctively (e.g., “Illustrative embodiment 1-4 is understood as illustrative embodiment 1, 2, 3, or 4.”).
Illustrative embodiment 1 is a method for minimally-invasive appendectomy comprising: inserting an inverter apparatus into an interior cavity of the appendix of a subject; engaging the inverter apparatus with an interior wall of the appendix; inverting the appendix by pulling the inverter apparatus into the cecum until the appendix is substantially inverted; positioning one or more closure devices around a base portion of the inverted appendix; closing the base portion of the inverted appendix with the one or more closure devices; positioning a separation apparatus at the base portion of the inverted appendix adjacent to the one or more closure devices; separating the inverted appendix from the cecum with the separation apparatus; and removing the separated appendix from the subject.
Illustrative embodiment 2 is the method of any preceding or subsequent illustrative embodiment, wherein the inverter apparatus comprises a plurality of arms.
Illustrative embodiment 3 is the method of any preceding or subsequent illustrative embodiment, wherein each of the plurality of arms comprise a curved end.
Illustrative embodiment 4 is the method of any preceding or subsequent illustrative embodiment, wherein the inverter apparatus engages the interior wall using negative pressure.
Illustrative embodiment 5 is the method of any preceding or subsequent illustrative embodiment, wherein the closure device comprises at least one of a slip knot, a band, a polymeric zipper tie, or combination thereof.
Illustrative embodiment 6 is the method of any preceding or subsequent illustrative embodiment, wherein the base of the inverted appendix is substantially aligned with an outer wall of the cecum.
Illustrative embodiment 7 is the method of any preceding or subsequent illustrative embodiment, wherein the separation apparatus comprises a metal wire.
Illustrative embodiment 8 is the method of any preceding or subsequent illustrative embodiment, wherein the separation apparatus uses electromagnetic radiation at frequencies between 350-500 kHz.
Illustrative embodiment 9 is the method of any preceding or subsequent illustrative embodiment, wherein the separation apparatus uses electromagnetic radiation at wavelengths between 800-1500 nm.
Illustrative embodiment 10 is the method of any preceding or subsequent illustrative embodiment, wherein the separated appendix remains engaged by the inverter apparatus.
Illustrative embodiment 11 is the method of any preceding or subsequent illustrative embodiment, further comprising accessing the cecum through the rectum.
Illustrative embodiment 12 is the method of any preceding or subsequent illustrative embodiment, further comprising: prior to removing the inverted appendix from the subject, positioning a size reduction apparatus around the inverted appendix in the cecum; and engaging the size reduction apparatus to reduce the size of the appendix to facilitate removal of the inverted appendix from the subject.
Illustrative embodiment 13 is the method of any preceding or subsequent illustrative embodiment, wherein a cross-section of the inverted appendix is reduced to a width of 24 French or less.
Illustrative embodiment 14 is the method of any preceding or subsequent illustrative embodiment, wherein a cross-section of the inverted appendix is reduced.
Illustrative embodiment 15 is the method of any preceding or subsequent illustrative embodiment, wherein the size reduction apparatus comprises articulating arms.
Illustrative embodiment 16 is the method of any preceding or subsequent illustrative embodiment, wherein the size reduction apparatus further comprises a metallic or polymeric mesh.
Illustrative embodiment 17 is the method of any preceding or subsequent illustrative embodiment, wherein the mesh comprises stainless steel, a cobalt chrome alloy, a nickel aluminum alloy, or a nickel titanium alloy.
Illustrative embodiment 18 is the method of any preceding or subsequent illustrative embodiment, wherein the size reduction apparatus comprises a cutting device.
Illustrative embodiment 19 is the method of any preceding illustrative embodiment, wherein the size reduction apparatus comprises an acid or base.
Illustrative embodiment 20 is a medical device comprising: a head comprising at least one arm extending outwardly from a first position of the head, wherein the at least one arm comprises at least one of a hooked, pointed, jagged, or rounded tip; and a handle connected to a second position of the head.
Illustrative embodiment 21 is the medical device of any preceding or subsequent illustrative embodiment, wherein the head comprises a plurality of arms.
Illustrative embodiment 22 is the medical device of any preceding or subsequent illustrative embodiment, wherein the plurality of arms move in a lateral direction at the head.
Illustrative embodiment 23 is the medical device of any preceding or subsequent illustrative embodiment, wherein the at least one arm is articulating.
Illustrative embodiment 24 is the medical device of any preceding or subsequent illustrative embodiment, wherein the handle further comprises a lever to articulate the at least one arm.
Illustrative embodiment 25 is the medical device of any preceding or subsequent illustrative embodiment, wherein the device comprises stainless steel, a cobalt chrome alloy, a nickel aluminum alloy, or a nickel titanium alloy.
Illustrative embodiment 26 is the medical device of any preceding or subsequent illustrative embodiment, wherein the device comprises a polymeric coating.
Illustrative embodiment 27 is the medical device of any preceding or subsequent illustrative embodiment, further comprising a metallic or polymeric mesh.
Illustrative embodiment 28 is the medical device of any preceding illustrative embodiment, wherein the mesh comprises stainless steel, a cobalt chrome alloy, a nickel aluminum alloy, or a nickel titanium alloy.
Illustrative embodiment 29 is a kit comprising: an inverter apparatus; one or more closure devices; and a separation apparatus.
Illustrative embodiment 30 is the kit of any preceding or subsequent illustrative embodiment, further comprising a size reduction apparatus.
Illustrative embodiment 31 is the kit of any preceding or subsequent illustrative embodiment, further comprising a catheter.
Illustrative embodiment 32 is a kit comprising: the medical device of any preceding embodiment; one or more closure devices; and a separation apparatus.

1. A method for minimally-invasive appendectomy comprising:
inserting an inverter apparatus into an interior cavity of the appendix of a subject;
engaging the inverter apparatus with an interior wall of the appendix;
inverting the appendix by pulling the inverter apparatus into the cecum until the appendix is substantially inverted;
positioning one or more closure devices around a base portion of the inverted appendix;
closing the base portion of the inverted appendix with the one or more closure devices;
positioning a separation apparatus at the base portion of the inverted appendix adjacent to the one or more closure devices;
separating the inverted appendix from the cecum with the separation apparatus; and
removing the separated appendix from the subject.

2. The method of claim 1, wherein the inverter apparatus comprises a plurality of arms.

3-5. (canceled)

6. The method of claim 1, wherein the base of the inverted appendix is substantially aligned with an outer wall of the cecum.

7. The method of claim 1, wherein the separation apparatus comprises a metal wire.

8. The method of claim 1, wherein the separation apparatus uses electromagnetic radiation at frequencies between 350-500 KHz.

9. The method of claim 1, wherein the separation apparatus uses electromagnetic radiation at wavelengths between 800-1500 nm.

10. The method of claim 1, wherein the separated appendix remains engaged by the inverter apparatus.

11. (canceled)

12. The method of claim 1, further comprising:
prior to removing the inverted appendix from the subject, positioning a size reduction apparatus around the inverted appendix in the cecum; and
engaging the size reduction apparatus to reduce the size of the appendix to facilitate removal of the inverted appendix from the subject.

13. The method of claim 12, wherein a cross-section of the inverted appendix is reduced to a width of 24 French or less.

14. (canceled)

15. The method of claim 12, wherein the size reduction apparatus comprises articulating arms.

16. The method of claim 15, wherein the size reduction apparatus further comprises a metallic or polymeric mesh.

17. (canceled)

18. The method of claim 12, wherein the size reduction apparatus comprises a cutting device.

19. (canceled)

20. A medical device comprising:
a head comprising at least one arm extending outwardly from a first position of the head, wherein the at least one arm comprises at least one of a hooked, pointed, jagged, or rounded tip; and
a handle connected to a second position of the head.

21. The device of claim 20, wherein the head comprises a plurality of arms.

22. (canceled)

23. The device of claim 20, wherein the at least one arm is articulating.

24. The device of claim 23, wherein the handle further comprises a lever to articulate the at least one arm.

25. The device of claim 20, wherein the device comprises stainless steel, a cobalt chrome alloy, a nickel aluminum alloy, or a nickel titanium alloy.

26-28. (canceled)

29. A kit comprising:
an inverter apparatus;
one or more closure devices; and
a separation apparatus.

30. The kit of claim 29, further comprising a size reduction apparatus.

31. The kit of claim 29, further comprising a catheter.

32. (canceled)
